(19) United States
(12) Patent Application Publication
(10) Pub. No.: US 2024/0285917 A1
(43) Pub. Date: Aug. 29, 2024

(54) RADIO TRANSMITTERS AND ANTENNA ASSEMBLIES FOR MEDICAL SYSTEMS

(71) Applicant: Shifamed Holdings, LLC, Campbell, CA (US)

(72) Inventors: Peter Andriola, Castro Valley, CA (US); Brian Fahey, Menlo Park, CA (US); Shahn Sage, Clear Lake, MN (US); Eric N. Rudie, Maple Grove, MN (US)

(21) Appl. No.: 18/572,141

(22) PCT Filed: Jun. 24, 2022
(86) PCT No.: PCT/US2022/034995

§ 371 Date: Dec. 19, 2023

Related U.S. Application Data
(60) Provisional application No. 63/215,309, filed on Jun. 25, 2021.

(51) Int. Cl. A61M 27/00

(52) U.S. Cl. CPC A61M 27/002; A61M 2205/0266; A61M 2205/04; A61M 2205/3303; A61M 2205/3327; A61M 2205/3592; A61M 2205/50; A61M 2205/8206; A61M 2205/8243; A61M 2210/125

(57) ABSTRACT

The present technology is directed to implantable medical devices including one or more communication elements configured to transmit and/or receive communication signals. The one or more communication elements can be coupled to one or more components of the device. The communication signals can be used to control, transmit, and/or to receive information from one or more of the components. The communication element(s) can additionally be coupled to a power source and used to transfer power to the components, and/or to transfer operational signals from a first component to a second component. The communication element(s) can be configured to perform a first function at a first (e.g., high) frequency or frequency range, and to perform a second function at a second (e.g., low) frequency or frequency range.

### CROSS-REFERENCE TO RELATED APPLICATION(S)

This application claims the benefit of U.S. Provisional Application No. 63/215,309, filed Jun. 25, 2021, the disclosure of which is incorporated by reference herein in its entirety.

### TECHNICAL FIELD

The present technology generally relates to implantable medical devices and systems and, in particular, to implantable medical devices and systems having radio transmitters and antenna assemblies.

### BACKGROUND

Implantable devices and systems are utilized in modern medicine to provide a host of diagnostic and/or therapeutic benefits. For example, implantable shunting systems are widely used to treat a variety of patient conditions by shunting fluid from a first body region/cavity to a second body region/cavity. The flow of fluid through the shunting systems is primarily controlled by the pressure gradient across the shunt lumen and the geometry (e.g., size) of the shunt lumen. One challenge with conventional shunting systems is selecting the appropriate geometry of the shunt lumen for a particular patient. A lumen that is too small may not provide enough therapy to the patient, while a lumen that is too large may create new issues in the patient. Despite this, most conventional shunts cannot be adjusted once they have been implanted. Accordingly, once the system is implanted, the therapy provided by the shunting system cannot be adjusted or titrated to meet the patient's individual needs.

As a result of the above, shunting systems with adjustable lumens have recently been proposed to provide a more personalized or titratable therapy. Such systems enable clinicians to titrate the therapy to an individual patient's needs, as well as adjust the therapy over time as the patient's disease changes. Some adjustable shunting systems include radio transmitters and antenna assemblies that allow clinicians to non-invasively communicate with the systems. It would be desirable to have a diverse set of communication options available to non-invasively communicate with an implanted device—for example, to provide energy to a device, to at least partially control the operation of the device, to extract information from the device, etc. However, previously disclosed devices and methods have challenges with accommodating the space and power requirements associated with the components required to achieve this diverse and multifaceted system communication paradigm, and often add complexity to the system that increases the system's overall size and weight and creates elevated risk of system failure.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIG. 1 is a schematic illustration of an interatrial device implanted in a heart of a patient and configured in accordance with select embodiments of the present technology.

FIG. 2A is a perspective view of a first device coupled to a second device via a communication element configured in accordance with select embodiments of the present technology.

FIG. 2B is a side cross-sectional view of the communication element of FIG. 2B.

FIG. 3 is a schematic illustration of a system including a communication element and configured in accordance with select embodiments of the present technology.

### DETAILED DESCRIPTION

The present technology is directed to implantable medical devices including one or more communication elements configured to transmit and/or receive communication signals from a source external to the implanted device (e.g., from a source external to a patient, from a separate device implanted within the patient, etc.). The communication element(s) can be operably coupled to one or more components of the device, such as an actuation element, an engine, a data storage/memory component, an energy storage component, a microcontroller, or a sensor. Accordingly, the communication element(s) can be used to transmit and/or receive signals between the implanted device and/or a device or user (e.g., a clinician) external to the patient. In at least some embodiments of the present technology, the communication elements can be used to transmit and/or receive signals related to the control of (e.g., operations, commands, etc.) the implanted device. In at least some embodiments of the present technology, the communication elements can be used to transmit and/or receive information (e.g., data, sensor measurements, device information, etc.) to or from one or more of the device components. In at least some embodiments, the communication element(s) can additionally be electrically coupled to a power source/energy storage component and used to transfer energy to device components (e.g., from the power source to a sensor), and/or to transfer operational signals from a first device component to a second device component (e.g., from a microcontroller to an engine). In at least some embodiments, the communication element(s) can be configured to perform a first function at a first (e.g., relatively higher) frequency or frequency range, and to perform a second function at a second (e.g., relatively lower) frequency or frequency range. For example, in the first frequency range, the communication element(s) can be configured to transmit or transfer a communication signal, and in the second frequency range, the communication element(s) can be configured to transfer energy and/or operational signals. In some embodiments the communication element(s) can perform the first and second functions at a same or different time, e.g., coextensively and/or independently of each other.

In one embodiment, the one or more communication elements are implemented in a cardiovascular treatment device such as an interatrial shunt or implantable pressure sensor. The shunt for example, may be configured for shunting fluid between a first body region (e.g., a left atrium) and a second body region (e.g., a right atrium) of a patient. The system further includes a shunting element having a lumen extending therethrough that is configured to fluidly couple the first and second body regions when the shunting element is implanted in the patient. The system can also include an actuation element (e.g., a shape memory actuation element) configured to adjust a geometry of the lumen to change the flow of fluid therethrough. Examples of an actuation element for modifying the shunt are described in U.S. patent application Ser. Nos. 16/840,108 and 17/016,192, the entire contents of which are incorporated by reference herein for all purposes. The system can further include a communication element for transmitting and/or receiving communication signals. The communication element can be operably coupled to the actuation element such that a clinician can send a communication signal to the communication element to directly (e.g., via supplying energy) or indirectly (e.g., by providing instruction to a microprocessor that subsequently activates another aspect of the system) adjust the geometry of the lumen. In some embodiments, the communication element can be operably coupled to one or more sensors, engines, storage/memory components, energy storage components, microcontrollers, and/or other components of the device. For example, in at least some embodiments the communication element can be operably coupled to a pressure sensor positioned in the first body region such that the communication element can transmit pressure measurements from the pressure sensor to a user (e.g., clinician). In other embodiments, the communication element can be operably coupled to a memory and/or a microprocessor configured to store information (e.g., pressure measurements) from a pressure sensor, and the stored information can be transmitted from the memory and/or the microprocessor to a user (e.g., clinician) via the communication element. Additionally, in at least some embodiments the communication element can be coupled to a power source of the device such that the communication element can transmit information related to the power source (e.g., power level, charge status, etc.). Moreover, in at least some embodiments, the communication element can be configured to transfer energy from the power source/energy storage component to one or more device components, such as the pressure sensor. Furthermore, in at least some embodiments the communication element can be configured to transfer one or more operational signals, e.g., from a first component to a second component.

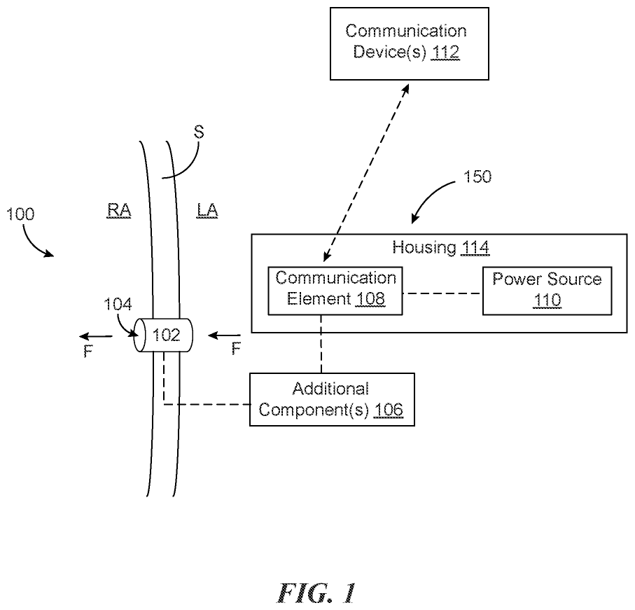

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to FIGS. 1-3.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Throughout this specification, “power” and “energy” are used somewhat interchangeably. One of ordinary skill in the art will appreciate, however, that power and energy are not always equivalent. For example, a battery can viewed as both an “energy” storage or “power” storage component. However, “power” may also refer to a rate at which stored energy is discharged. Thus, one of ordinary skill in the art will understand that the terms “power” and “energy” may have a same or different meaning in various places throughout this specification based the context and/or manner in which these terms are used.

FIG. 1 is a schematic illustration of a medical device system 100 (“the system 100”) including one or more communication elements configured in accordance with an embodiment of the present technology. The system 100 includes a shunting element 102 defining a lumen 104 therethrough. In the illustrated embodiment, the shunting element 102 is implanted across a septal wall S in a patient's heart, although the shunting element 102 can be implanted in other regions of the body to fluidly connect any two body regions. When implanted across the septal wall S, the system 100 can fluidly connect a left atrium LA and a right atrium RA of the heart via the lumen 104. Accordingly, when the shunting element 102 is implanted in the septal wall S of some patients, blood can flow between the left atrium LA to the right atrium RA via the lumen 104 (as shown by arrows F). The system 100 can further include one or more additional components 106 that can be coupled to the shunting element 102. Some of the additional component(s) 106 can include features implanted with the shunting element 102 that require power to operate (i.e., active components). For example, active components can include one or more actuation elements (e.g., for adjusting a geometry or other characteristic of the shunting element 102), an engine, a storage/memory, a microcontroller, a sensor (e.g., for measuring one or more physiological parameters and/or one or more parameters of the system 100), and other components. The shunting element 102 can include additional features not shown in FIG. 1, including inactive components such as a frame, membrane, or the like.

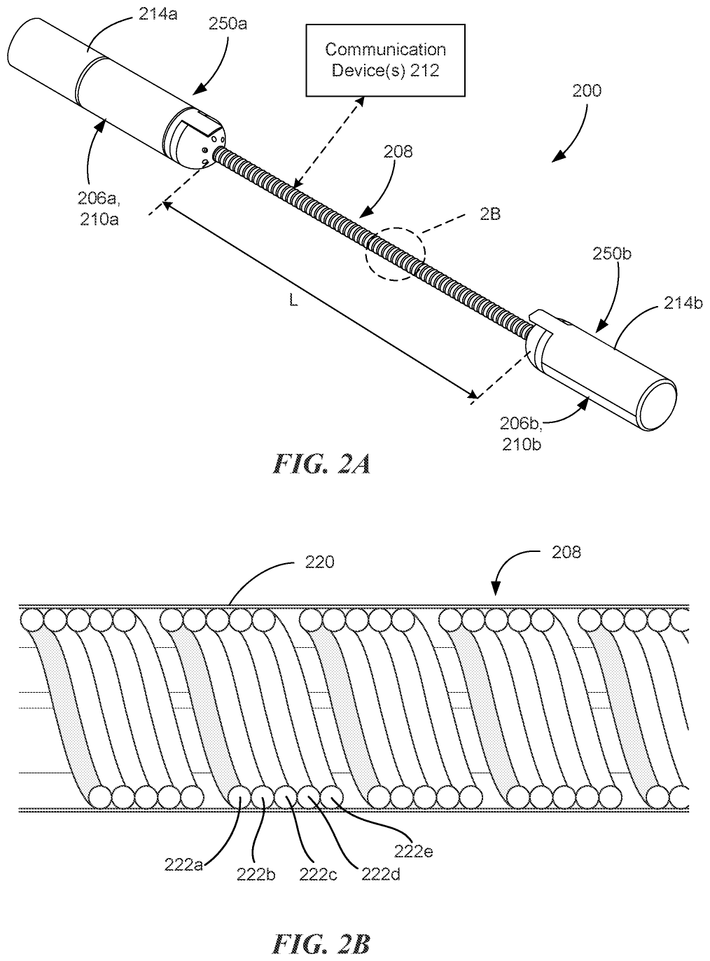

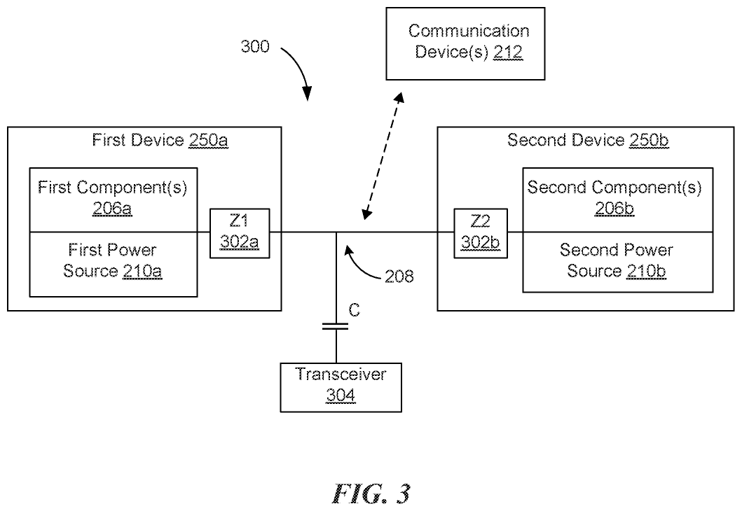

The system 100 can further include a communication element 108 and a power source 110. As explained in greater detail below with reference to FIGS. 2A-3, the communication element 108 can couple (e.g., electrically, operatively, communicatively, etc.) the power source 110 and the additional component(s) 106, such that communication element 108 can transfer or convey energy from the power source 110 to the additional component(s) 106. Additionally, the communication element 108 can be configured to transmit and/or receive communication signals (e.g., information, data, commands, etc.). For example, the communication element 108 can transmit a communication signal from the additional component(s) 106 and/or the power source 110 to a device or user (e.g., a clinician) external to a patient. In at least some embodiments, the communication element 108 can transmit operational signals from a first device component (e.g., a sensor) to a second device component (e.g., a motor, a microprocessor, an actuation element, a shape memory actuation element, etc.). In at least some embodiments, the communication element 108 can include components such as wires, antennae, transmitters, transceivers, and/or any other suitable components.

The power source 110 can be or include a battery (e.g., a lithium-ion battery, a lithium primary battery, a zinc-ion battery, an alkaline battery, and/or any other suitable battery or cell), a supercapacitor, a capacitor, and/or one or more inductive elements configured to receive and/or store energy from an energy transmission device. Examples of systems that implement power-receiving inductive elements are described in U.S. Provisional Patent App. No. 63/093,073, filed Oct. 16, 2020, the entire contents of which is incorporated by reference herein for all purposes.

In some embodiments, one or more communication devices 112 can communicate (e.g., interact, control, monitor, exchange data, transfer data, etc.) with the system 100. The communication device(s) 112 can be positioned external to the system 100, e.g., such that a user (e.g., a clinician) can use the communication device(s) 112 to communicate with the system 100 when the system 100 is implanted in a patient. In some embodiments the communication device(s) 112 can be positioned inside of the patient but be separate from the system 100 and/or one or more of the components of the system 100 (e.g., the shunting element 102, the additional component(s) 106, the housing 114, etc.). In some such embodiments, the internally-located communication device(s) 112 can be positioned in a same or different anatomical region of the body relative to system 100. In some embodiments, multiple communication device(s) 112 can be included, and the communication element 108 can be configured to communicate with each of the multiple communication devices 112 directly, and/or the communication element 108 can be configured to communicate with a first communication device as part of a communication chain that includes one or more additional communication devices (i.e., the first communication device communicates with both the system 100, via the communication element 108, and the additional communication device(s), but the additional communication device(s) communicate with the first communication device and does not communicate with system 100 directly).

The communication device(s) 112 can be configured to communicate with the communication element 108, including transmitting communication signals to the communication element 108 and/or receiving communication signals from the communication element 108. The communication device(s) 112 can include any device or system external to the implant that is capable of wirelessly transmitting communication signals to an implanted component. For example, the communication device 112 and/or the communication element 108 can be configured to transmit and/or receive radiofrequency (RF) energy, microwave frequency energy, other forms of electromagnetic energy, ultrasonic energy, thermal energy, or other types of energy in accordance with techniques known to those of skill in the art. In some embodiments, the communication device 112 and/or the communication element 108 may operate in a frequency range between about 1 MHz and about 1 GHz, such as between about 900 MHz and about 930 MHz (e.g., 900 MHz, 901 MHZ, 902 MHZ, 903 MHz, 904 MHZ, 905 MHz, etc.), between about 910 MHz and about 920 MHz, and/or about 915 MHz, although other frequencies are possible.

In at least some embodiments, one or more aspects of the system 100 can be combined in a device 150. The device 150 can include a can or housing 114 configured to contain (e.g., house, carry, encapsulate, etc.) one or more elements of the system 100. For example, in at least some embodiments the housing 114 can contain the communication element 108, the power source 110, and/or one or more of the additional components(s) 106 (e.g., one or more sensors, microcontroller, engines, memory, and/or actuation elements). In at least some embodiments, one or more of the elements of the systems 100 can have separate or individual housings.

The housing 114 and/or the device 150 can be placed within a catheter (not shown) and used to deliver the shunting element 102, communication element 108, the power source 110, and/or one or more of the additional components 106 to a target site within the patient. The housing 114 and/or the device 150 can be coupled (e.g., mechanically) to the shunting element 102, at least partially embedded in the septal wall S, and/or positioned at least partially in the first body cavity (e.g., the left atrium) and/or the second body cavity (e.g., the right atrium). In at least some embodiments, the system 100 can include a plurality of devices, wherein each of the devices can include a housing, and one or more of the communication element 108, the power source 110, and/or the additional component(s) 106 can be distributed and/or shared amongst the plurality of devices. For example, in at least some embodiments the communication element 108 can couple (e.g., mechanically, electrically, operatively, communicatively, etc.) a first device to one or more other devices.

As will be understood by one of skill from the description herein, the communication element 108 can be formed from a variety of materials including, but not limited to metals, alloys, and any other suitable material. The shape and configuration of the communication element 108 may be determined based on the material properties, delivery technique, and/or requirements of the application. The configuration of the communication element 108 will be described in more detail below.

FIGS. 2A and 2B illustrate view of a system 200 configured in accordance with select embodiments of the present technology. The system 200 and/or one or more components thereof can be generally similar or the same as the system 100 of FIG. 1. Accordingly, like numbers are used to designate like components (e.g., communication element 208 versus communication element 108 of FIG. 1). In FIGS. 2A and 2B a number of system aspects have been omitted for clarity, and the following discussion of FIGS. 2A and 2B will be limited to those features that are different from system 100 and/or are necessary for context.

FIG. 2A illustrates a perspective view of a first subassembly or device 250a and a second subassembly or device 250b configured in accordance with select embodiments of the present technology. The first device 250a can include one or more first component(s) 206a and/or a first power source 210a positioned within a first can or housing 214a. Similarly, the second device 250b can include one or more second component(s) 206b and/or a second power source 210b positioned within a second can or housing 214b.

The first device 250a can be coupled (e.g., mechanically, electrically, communicatively, operably, etc.) to the second device 250b via a communication element 208. As described previously, the communication element 208 can be configured to transmit and/or receive communication signals from a communication device(s) 212. Additionally, and as described previously, the communication element 208 can be configured to connect the one or more first component(s) 206a and/or the first power source 210a to the one or more second component(s) 206b and/or the second power source 210b, e.g., to transfer power from a power source to one or more components and/or to transfer operational signals from a first component to a second component.

The communication element 208 can have a length L such that the first device 250a and the second device 250b are spaced apart from each other by the length L when arranged in the configuration shown in FIG. 2A. In some embodiments, the communication element 208 can be flexible and be bent or otherwise arranged such that the first device 250a and the second device 250b can be positioned closer to one another than the length L while retaining a communication path distance between the devices equal to the length L. In at least some embodiments, the length L can correspond to a wavelength at which the communication element 208 is configured to transmit and/or receive communication signals from the communication device(s) 212. In at least some embodiments, the length L can be selected to account for alterations of one or more characteristics (e.g., wavelength, frequency, etc.) of the communication signals that is associated with their travel through one or more media (e.g., blood) or environments (e.g., the left atrium and/or right atrium), e.g., the media/environment in which the system 200 will be operating. For example, in at least some embodiments, the length L of the communication element 208 corresponds to the electromagnetic wavelength in blood of an about 915 MHz radiofrequency signal, or any of the other frequencies or frequency ranges described herein. Accordingly, the length L can be selected such that the communication element 208 can be configured to operate as an electromagnetic antenna and/or transmitter at about a 915 MHz signal, or any of the other frequencies or frequency ranges described above (e.g., between about 900 MHz and about 930 MHz, between about 910 MHz and about 920 MHz, etc.).

In some embodiments, insulating materials with a particular dielectric constant may be incorporated in the communication element 208 in order change the frequency that a particular length L corresponds to, and thereby allow for additional selective adjustments for sizing of the system 200. For example, an insulating material such as polyurethane, silicone rubber, or polytetrafluoroethylene with a dielectric constant in the range of 1-5 (e.g., 1.5, 2.5, 3.5, or 4.5) could be added as a jacket on the communication element 208. In such an arrangement, the effective length L (which, as discussed above, corresponds to a wavelength at which the communication element 208 is configured to transmit and/or receive communication signals) can be increased by about 1-20%. In other embodiments, the effective length L of the communication element 208 corresponds to the acoustic wavelength in blood of an about 50 kHz ultrasonic signal. Accordingly, the effective length L can be selected such that the communication element 208 can be configured to operate as an acoustic resonator at about a 50 kHz signal.

In some embodiments, the length L can be between about 0.5 cm and about 20 cm, such as at least 1 cm, 2 cm, or 4 cm or any other suitable length. In some embodiments, the communication element 208 has a total length L but additionally or alternatively has one or more sub-sections smaller than the total length L configured to operate as an antenna and/or transmitter. These one or more subsections can correspond different wavelengths of communication signals. Accordingly, such embodiments can enable multi-frequency and/or multi-source communications between a communication element 208 and communication device(s) 212 and/or other components. In some embodiments, communication element 208 can operate both as an electromagnetic antenna and an acoustic resonator.

FIG. 2B is a side cross-sectional view of region 2B of communication element 208 of FIG. 2A. In the illustrated embodiment, the communication element 208 includes one or more leads or wires 222 surrounded by an insulating layer 220. Each of the wires 222 can couple the one or more first component(s) 206a and/or the first power source 210a to the one or more second component(s) 206b and/or the second power source 210b (FIG. 2A). Additionally, at least one of the wires 222 can be configured to transmit and/or receive communication signals from the communication device(s) 212. For example, in the illustrated embodiment, the first wire 222a is configured to transmit power and/or operational signals between the first and second devices 250a-b, and can be additionally configured to transmit and/or receive communication signals between the first and/or second devices 250a-b and the external communication device(s) 212. As described in greater detail below, the function or operation performed by the first wire 222a can vary based on a frequency of a signal provided to the first wire 222a.

In the illustrated embodiment, the communication element 208 includes a first wire 222a, a second wire 222b, a third wire 222c, a fourth wire 222d, and a fifth wire 222e (referred to collectively as “the wires 222”). In other embodiments, the communication element 208 can include more or fewer wires 222, such as one, two, three, four, six, seven, eight, or more wires 232. Additionally, in the illustrated embodiments the wires 222 are coiled or wrapped in a helical configuration. In other embodiments, however, the wires 222 can be bundled, packaged, wrapped, and/or otherwise stored in any suitable configuration.

FIG. 3 is a schematic illustration of a system 300 including the communication element 208 and configured in accordance with select embodiments of the present technology. Referring to FIGS. 2A-3 together, the system 300 can include a first element 302a having a first impedance Z1, a second element 302b having a second impedance Z2, and a capacitor C. In some embodiments, the transceiver 304 and capacitor C can be part of the first device 250a or the second device 250b. In such embodiments, the coupling between the transceiver 304 and the communication device(s) 212 can be incorporated in the first components 206a and/or the second components 206b. In other embodiments, there may be a first transceiver 304 and capacitor C in the first device 250a and a second transceiver 304 and capacitor C in the second device 250b. In such embodiments, the first and second transceivers can be coupled through respective capacitors C to either the same or different elements within the communication device 212. In other embodiments, the communication element 208 can couple (e.g., electrically, mechanically, etc.) the first element 302a, the second element 302b, and the capacitor C. Each of the first and second elements 302a-b can have a relatively high impedance at high frequencies, and a relatively low impedance at low frequencies. The first and second elements 302 a-b can include, for example, inductors, lossy ferrites, a combination thereof, or any other suitable component with these properties as would be known to one skilled in the art. In contrast, the capacitor C can have a relatively low impedance at high frequencies, and a relatively high impedance at low frequencies. In some embodiments, the capacitor C can be replaced with other elements having similar frequency properties, for example a gyrator connected to an inductor, or any other suitable elements and/or circuits with these properties as would be known to one skilled in the art.

In the illustrated embodiment, the first device 250 includes the first element 302a, and the first element 302a can be coupled to the first component(s) 206a and/or the first power source 210a. Similarly, in the illustrated embodiment the second device 250b includes the second element 302b, and the second element 302b can be coupled to the second component(s) 206b and/or the second power supply 210b. Additionally, the capacitor C can be coupled (e.g., electrically) to one or more radio or transceiver elements 304. The transceiver element(s) 304 can be configured to transmit and/or receive communication signals via the communication element 208, e.g., to communicate with a communication device(s) 212. The transceiver element(s) 304 can be coupled to (e.g., electrically, mechanically, etc.) and/or part of the first and/or second devices 250a-b. For example, in at least some embodiments, the first device 250a includes a first transceiver element coupled to the corresponding first component(s) 206a and/or first power source 210a, and the second device 250b includes a second transceiver element coupled to the corresponding second component(s) 206b and/or second power source 210b. In at least some embodiments, the transceiver element(s) 304 can be in communication (e.g., electrically coupled) with the first and/or second devices 250a-b, and/or one or more components thereof, such that the transceiver element(s) 304 can be used to transmit information related to the first and/or second devices 250a-b to the communication device(s) 212.

The first and second component(s) 206a-b and the first and second power sources 210a-b can be configured to transmit and/or receive energy and/or operational signals at relatively low frequencies. For example, the energy and/or operational signals can include frequencies in a range between about 0 MHz (e.g., for DC power) and about 500 MHz, such as between about 1 MHz and about 15 MHz (e.g., 1 MHz, 2 MHZ, 3 MHz, 4 MHz, 5 MHZ, 6 MHZ, etc.), although other frequencies are possible. The transceiver element 304 can be configured to transmit and/or receive communication signals at relatively high frequencies. For example, the communication signals can include frequencies such as between about 900 MHz and about 930 MHz (e.g., 900 MHz, 901 MHZ, 902 MHZ, 903 MHz, etc.), between about 910 MHz and about 920 MHZ, and/or about 915 MHZ, although other frequencies are possible. In some embodiments, the operational signals can include frequencies in a range between about 0 Hz and the communication signals. Accordingly, based on the relative impedances of the first element 302a, the second element 302b, and the capacitor C, the communication element 208 can be configured to perform a first function at a first (e.g., relatively higher) frequency or frequency range, and to perform a second function at a second (e.g., relatively lower) frequency or frequency range. For example, the impedances of the first and second elements 302a-b can at least partially prevent or impede first (e.g., high) frequency signals from reaching the first and/or second devices 250a-b. Accordingly, at the first frequencies the communication element 208 can be configured to perform a first function, e.g., to operate as an antenna for the transceiver 304 to communicate with the communication devices 212. Similarly, the impedance of the capacitor C can at least partially prevent or impede second (e.g., low) frequency signals from reaching the transceiver 304. Accordingly, at the second frequencies the communication element 208 can be configured to perform a second function, e.g., to transfer power and/or operational signals between the first and second devices 250a-b and/or one or more components thereof.

The system 300 can be configured such that the communication element 208 can perform the first and second functions at a same or different time. For example, the difference between the first and second frequencies or frequency ranges can be such that the communication element 208 can communicate with the communication device(s) 212 and transfer energy and/or operational signals between the first and second devices 250a simultaneously. This can include, for example, a difference of at least 0 Hz, 1 Hz, 10 Hz, 100 Hz, 1 kHz, 1 MHz, 1 GHz and/or any other suitable difference. Accordingly, the communication element 208 can be configured to operate in response to a plurality of signals concurrently, including a first higher frequency signal and a second relatively lower frequency signal.

As one skilled in the art will appreciate, communication elements configured in accordance with the present technology (e.g., communication element 208) can be incorporated in other electrical systems beyond those illustrated in FIG. 3. For example, the communication element (s) 208 can be incorporated into a conventional RLC circuit that provides energy to an energy storage device (e.g., a supercapacitor, a battery, etc.), which can subsequently release the stored energy to power an active component (e.g., motor, shape memory actuation element, sensor, etc.).

Communication elements configured in accordance with the present technology offer a number of advantages over technologies that are presently available. As described previously, traditional systems typically utilize radio transceivers having antennas disposed separately from other components of the system. Such an arrangement can add complexity to the system and increase the system's overall size and weight. In contrast with such conventional systems, devices configured in accordance with the present technology, which incorporates the antenna into a communication device which can also provide other aspects of the system (e.g., structural support, anatomical anchoring, and/or communication between a plurality of devices in the system) can be relatively smaller in size. This is expected to benefit patients by leaving more room around the implant (e.g., more room on a septal wall) to enable future procedures that require septal access or crossing (e.g., pulmonary vein ablation, mitral valve procedures, left atrial appendage closures, etc.), and/or allow for a smaller implant and, therefore, a smaller catheter delivery size, which increases the safety profile for patients. Moreover, devices configured in accordance with the presently disclosed technology are expected to be more robust to failure.

As will also be appreciated, various components of the systems described herein can be omitted without deviating from the scope of the present technology. Likewise, additional components not explicitly described above may be added to the systems without deviating from the scope of the present technology. Moreover, the communication elements described herein can be incorporated into other types of implantable medical devices beyond cardiac shunts. Accordingly, the present technology is not limited to the configurations expressly identified herein, but rather encompasses variations and alterations of the described systems.

### EXAMPLES

Several aspects of the present technology are set forth in the following examples:

1. A system for shunting fluid between a first body region and a second body region of a patient, the system comprising:
a shunting element configured to be implanted in the patient;
an actuation element configured to adjust a geometry of the lumen; and
an electrical circuit including at least one communication element configured to—
   transmit and/or receive one or more communication signals to a communication device external to the patient at a first frequency range, and
   transfer power and/or one or more operational signals at a second frequency range different than the first frequency range.

2. The system of example 1 wherein the first frequency range is between about 910 MHz and about 920 MHz.

3. The system of example 2 wherein the communication element has a length that corresponds to the first frequency range.

4. The system of any one of examples 1-3 wherein the second frequency range is at least 1 Hz less than the first frequency range.

5. The system of any one of examples 1-3 wherein the second frequency range is at least 1 MHz less than the first frequency range.

6 The system of any one of examples 1-3 wherein the second frequency range is at least 1 GHz less than the first frequency range.

7. The system of any one of examples 1-6 wherein the communication element is configured to receive a plurality of signals simultaneously, and wherein the plurality of signals includes a first signal at the first frequency range and a second signal at the second frequency range.

8. The system of any one of examples 1-7, further comprising a first device and a second device, wherein the at least one communication element is configured to mechanically couple the first device and the second device.

9. The system of example 8 wherein the first device includes a first housing containing one or more first components and/or a first power source, and wherein the second device includes a second housing containing one or more second components and/or a second power source.

10. The system of example 9 wherein the communication element is configured to operably couple at least two of the following: (i) the one or more first components, (ii) the first power source, (iii) the one or more second components, and/or (iv) the second power source.

11. The system of example 10 wherein, at the second frequency range, the communication element is configured to transfer power and/or one or more operational signals between at least two of the following: (i) the one or more first components, (ii) the first power source, (iii) the one or more second components, and/or (iv) the second power source.

12. The system of any one of examples 1-11 wherein the at least one communication element includes a plurality of wires coiled or wrapped in a helical configuration.

13. The system of any one of examples 1-12 wherein:
the electrical circuit further includes a first element have a first impedance and coupled to the first device, a second element having a second impedance and coupled to the second device, and a capacitor coupled to a transceiver; and
the at least one communication element is coupled to the first element, the second element, and the capacitor.

14. An electrical circuit for use with an implantable medical device, the electrical circuit comprising:
a communication element operably coupled to the implantable medical device, wherein the communication element comprises at least one wire configured to:
transmit and/or receive one or more communication signals to an external communication device at a first frequency range, and
transfer power and/or one or more operational signals at a second frequency range.

15. The electrical circuit of example 14 wherein the first frequency range is between about 910 MHz and about 920 MHz.

16. The electrical circuit of example 14 or example 15 wherein the at least one wire has a length that corresponds to the first frequency range.

17. The electrical circuit of any one of examples 14-16 wherein the second frequency range is between about 0 Hz and the first frequency range.

18. The electrical circuit of any one of examples 14-16 wherein the second frequency range is 1 Hz less than the first frequency range.

19. The electrical circuit of any one of examples 14-16 wherein the second frequency range is 1 kHz less than the first frequency range.

20. The electrical circuit of any one of examples 14-16 wherein the second frequency range is 1 MHz less than the first frequency range.

21. The electrical circuit of any one of examples 14-16 wherein the second frequency range is 1 GHz less than the first frequency range.

22 The electrical circuit of any one of examples 14-21 wherein the at least one wire is configured to receive a plurality of signals simultaneously, wherein the plurality of signals includes a first signal at the first frequency range and a second signal at the second frequency range.

23. The electrical circuit of any one of examples 14-22, further comprising a first inductor coupled to a first device, a second inductor coupled to a second device, and a capacitor coupled to a transceiver, and wherein the at least one communication element is electrically coupled to the first inductor, the second inductor, and the capacitor.

24. An implantable medical device, comprising:
a plurality of wires, wherein the plurality of wires are configured to operably couple at least two of the following together: one or more first components, one or more second components, a first power source, and a second power source, and
wherein at least one of the wires is additionally coupled to a transceiver and configured to—
   transmit and/or receive one or more communication signals to an external communication device at a first frequency range, and
   transfer power and/or one or more operational signals between at least two of: (i) the one or more first components, (ii) the first power source, (iii) the one or more second components, and/or (iv) the second power source at a second frequency range.

25. The device of example 24 wherein the first frequency range is between about 910 MHz and about 920 MHz.

26. The device of example 24 or example 25 wherein the at least one of the wires has a length that corresponds to the first frequency range.

27. The device of any one of examples 24-26 wherein the at least one of the wires is configured to receive a plurality of signals simultaneously, and wherein the plurality of signals includes a first signal at the first frequency range and a second signal at the second frequency range.

28. The device of any one of examples 24-27 wherein the plurality of wires are arranged in a helical configuration.

29. A method, comprising:
transmitting, at a first frequency range, one or more communication signals from a transceiver of an implantable medical device to a communication device external to a patient; and
transmitting, at a second frequency range, one or more operational signals between a first component of the implantable medical device and a second component of the implantable medical device,
wherein transmitting at the first and second frequency ranges includes transmitting via a communication element.

30. The method of example 29 wherein the first component and/or second component includes at least one of the following: an actuation element, an engine, a microcontroller, a sensor, and a power source.

31. The method of example 29 or example 30 wherein the communication signals are first communication signals, and wherein the method further comprises receiving via the transceiver at the first frequency range, one or more second communication signals from the communication device.

32. The method of any one of examples 29-31 wherein the first frequency range is between about 910 MHz and about 920 MHz.

33. The method of any one of examples 29-32 wherein the communication element has a length corresponding to the first frequency range.

34. The method of any one of examples 29-33 wherein the communication element is configured to transmit at the first and second frequency ranges simultaneously.

### CONCLUSION

Embodiments of the present disclosure may include some or all of the following components: a battery, supercapacitor, or other suitable power source; a microcontroller, FPGA, ASIC, or other programmable component or system capable of storing and executing software and/or firmware that drives operation of an implant; memory such as RAM or ROM to store data and/or software/firmware associated with an implant and/or its operation; wireless communication hardware such as an antenna system configured to transmit via Bluetooth, WiFi, LoRa, Thread, Zigbee, UWB, or other protocols known in the art; energy harvesting means, for example a coil or antenna which is capable of receiving and/or reading an externally-provided signal which may be used to power the device, charge a battery, initiate a reading from a sensor, or for other purposes. Embodiments may also include one or more sensors, such as pressure sensors, impedance sensors, accelerometers, force/strain sensors, temperature sensors, flow sensors, optical sensors, cameras, microphones or other acoustic sensors, ultrasonic sensors, ECG or other cardiac rhythm sensors, SpO2 and other sensors adapted to measure tissue and/or blood gas levels, blood volume sensors, and other sensors known to those who are skilled in the art. Embodiments may include portions that are radiopaque and/or ultrasonically reflective to facilitate image-guided implantation or image guided procedures using techniques such as fluoroscopy, ultrasonography, or other imaging methods. Embodiments of the system may include specialized delivery catheters/systems that are adapted to deliver an implant and/or carry out a procedure. Systems may include components such as guidewires, sheaths, dilators, and multiple delivery catheters. Components may be exchanged via over-the-wire, rapid exchange, combination, or other approaches.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments. For example, although this disclosure has been written to describe devices that are generally described as being used to create a path of fluid communication between the left atrium and the right atrium, it should be appreciated that similar embodiments could be utilized for shunts between other chambers of the heart or for shunts in other regions of the body.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A system for shunting fluid between a first body region and a second body region of a patient, the system comprising:
a shunting element configured to be implanted in the patient;
an actuation element configured to adjust a geometry of the lumen; and
an electrical circuit including at least one communication element configured to—
transmit and/or receive one or more communication signals to a communication device external to the patient at a first frequency range, and
transfer power and/or one or more operational signals at a second frequency range different than the first frequency range.

2. The system of claim 1 wherein the first frequency range is between about 910 MHz and about 920 MHz.

3. The system of claim 2 wherein the communication element has a length that corresponds to the first frequency range.

4. The system of claim 1 wherein the second frequency range is at least 1 Hz less than the first frequency range.

5. The system of claim 1 wherein the second frequency range is at least 1 MHz less than the first frequency range.

6. The system of claim 1 wherein the second frequency range is at least 1 GHz less than the first frequency range.

7. The system of claim 1 wherein the communication element is configured to receive a plurality of signals simultaneously, and wherein the plurality of signals includes a first signal at the first frequency range and a second signal at the second frequency range.

8. The system of claim 1, further comprising a first device and a second device, wherein the at least one communication element is configured to mechanically couple the first device and the second device.

9. The system of claim 8 wherein the first device includes a first housing containing one or more first components and/or a first power source, and wherein the second device includes a second housing containing one or more second components and/or a second power source.

10. The system of claim 9 wherein the communication element is configured to operably couple at least two of the following: (i) the one or more first components, (ii) the first power source, (iii) the one or more second components, and/or (iv) the second power source.

11. The system of claim 10 wherein, at the second frequency range, the communication element is configured to transfer power and/or one or more operational signals between at least two of the following: (i) the one or more first components, (ii) the first power source, (iii) the one or more second components, and/or (iv) the second power source.

12. The system of claim 1 wherein the at least one communication element includes a plurality of wires coiled or wrapped in a helical configuration.

13. The system of claim 1 wherein:
the electrical circuit further includes a first element have a first impedance and coupled to the first device, a second element having a second impedance and coupled to the second device, and a capacitor coupled to a transceiver; and
the at least one communication element is coupled to the first element, the second element, and the capacitor.

14. An electrical circuit for use with an implantable medical device, the electrical circuit comprising:
a communication element operably coupled to the implantable medical device, wherein the communication element comprises at least one wire configured to:
transmit and/or receive one or more communication signals to an external communication device at a first frequency range, and
transfer power and/or one or more operational signals at a second frequency range.

15. The electrical circuit of claim 14 wherein the first frequency range is between about 910 MHz and about 920 MHz.

16. The electrical circuit of claim 14 wherein the at least one wire has a length that corresponds to the first frequency range.

17. The electrical circuit of claim 14 wherein the second frequency range is between about 0 Hz and the first frequency range.

18. The electrical circuit of claim 14 wherein the second frequency range is 1 Hz less than the first frequency range.

19. The electrical circuit of claim 14 wherein the second frequency range is 1 kHz less than the first frequency range.

20. The electrical circuit of claim 14 wherein the second frequency range is 1 MHz less than the first frequency range.

21. The electrical circuit of claim 14 wherein the second frequency range is 1 GHz less than the first frequency range.

22. The electrical circuit of claim 14 wherein the at least one wire is configured to receive a plurality of signals simultaneously, wherein the plurality of signals includes a first signal at the first frequency range and a second signal at the second frequency range.

23. The electrical circuit of claim 14, further comprising a first inductor coupled to a first device, a second inductor coupled to a second device, and a capacitor coupled to a transceiver, and wherein the at least one communication element is electrically coupled to the first inductor, the second inductor, and the capacitor.

24. An implantable medical device, comprising:
a plurality of wires, wherein the plurality of wires are configured to operably couple at least two of the following together: one or more first components, one or more second components, a first power source, and a second power source, and
wherein at least one of the wires is additionally coupled to a transceiver and configured to—
transmit and/or receive one or more communication signals to an external communication device at a first frequency range, and
transfer power and/or one or more operational signals between at least two of: (i) the one or more first components, (ii) the first power source, (iii) the one or more second components, and/or (iv) the second power source at a second frequency range.

25. The device of claim 24 wherein the first frequency range is between about 910 MHz and about 920 MHz.

26. The device of claim 24 wherein the at least one of the wires has a length that corresponds to the first frequency range.

27. The device of claim 24 wherein the at least one of the wires is configured to receive a plurality of signals simultaneously, and wherein the plurality of signals includes a first signal at the first frequency range and a second signal at the second frequency range.

28. The device of claim 24 wherein the plurality of wires are arranged in a helical configuration.

29. A method, comprising:
transmitting, at a first frequency range, one or more communication signals from a transceiver of an implantable medical device to a communication device external to a patient; and
transmitting, at a second frequency range, one or more operational signals between a first component of the implantable medical device and a second component of the implantable medical device,
wherein transmitting at the first and second frequency ranges includes transmitting via a communication element.

30. The method of claim 29 wherein the first component and/or second component includes at least one of the following: an actuation element, an engine, a microcontroller, a sensor, and a power source.

31. The method of claim 29 wherein the communication signals are first communication signals, and wherein the method further comprises receiving via the transceiver at the first frequency range, one or more second communication signals from the communication device.

32. The method of claim 29 wherein the first frequency range is between about 910 MHz and about 920 MHz.

33. The method of claim 29 wherein the communication element has a length corresponding to the first frequency range.

34. The method of claim 29 wherein the communication element is configured to transmit at the first and second frequency ranges simultaneously.
